(19) United States
(12) Patent Application Publication
(10) Pub. No.: US 2021/0171272 A1
(43) Pub. Date: Jun. 10, 2021

(54) First Aid Cabinet with Dispenser

(71) Applicant: Acme United Corporation, Fairfield, CT (US)

(72) Inventors: Brian S. Olschan, Madison, CT (US); Alan Peterson, Trumbull, CT (US)

(21) Appl. No.: 17/149,050

(22) Filed: Jan. 14, 2021

Related U.S. Application Data
(63) Continuation-in-part of application No. 16/704,244, filed on Dec. 5, 2019.

(51) Int. Cl. B65D 83/08; A61B 42/40; B65D 25/04

(52) U.S. Cl. CPC B65D 83/0817; B65D 25/04; B65D 83/0823; A61B 42/40

(57) ABSTRACT

A cabinet for first aid supplies employs a pair of shell sections which are pivotal between a closed position and an opened position. A frontal shell section defines one or more openings which communicate with a respective dispenser receptacle disposed inside of and carried by the frontal shell section. A stack of items is disposed in each dispenser receptacle and is engaged by an integrated leaf spring. An item may be expeditiously removed from either dispenser receptacle through the opening while the cabinet is closed. The openings and springs are configured to provide ready access to selected first aid items while protecting the items in an orderly fashion.

### CROSS-REFERENCE TO RELATED APPLICATION

This application is a continuation-in-part of U.S. patent application Ser. No. 16/704,244 filed on Dec. 5, 2019.

### BACKGROUND

This disclosure relates to cabinets which contain organized first aid supplies. More particularly, this disclosure relates to cabinets which are mountable to a wall and have a cover which pivots to provide access to first aid items.

For cabinets to which the present disclosure relates, a compact durable cabinet has a structure which protects first aid supplies and readily opens to provide access to the supplies. Various compartments are provided in the cabinet to organize the first aid supplies. The cabinet is also typically organized in a fashion which permits a ready location of a first aid item and facilitates inventory of the supplies.

### SUMMARY

Briefly stated, a cabinet comprises a base section and a cover section pivotally joined to the base section. The cover section is positionable between an opened and a closed position forming a housing. A receptacle is mounted to the cover section and accessible through the cover section in the closed position. The receptacle has a spring which is biased toward an interior surface of the receptacle.

The receptacle further receives a stack of first aid items and biases the stack to urge the stack toward a front portion of the cover section. A dispenser chute forms an opening through the cover section. An item of the stack is withdrawable through the chute while the cabinet is in the closed position.

A second receptacle is mounted to the cover section. The second receptacle is accessible through the front cover in the closed position. A spring biases the stack of first aid items received in the second receptacle to urge the stack toward the front panel portion of the cover section. A second dispenser chute forms an opening in the cover section so that the first aid item in the second receptacle is also withdrawable through the opening while the cabinet is closed.

The base section and the cover section cooperatively form a recess and a handle. The base section has a rear panel which defines a pair of keyhole openings. A pivotal locking plate secures the cabinet in a closed position. The cover section may be transparent or semi-transparent.

A first aid cabinet comprises a base section and a front cover section pivotally joined to the base section. The cover section is positionable between an opened and a closed position forming a housing for first aid supplies. A dispenser receptacle is carried by the front cover section and accessible through the cover section in the closed position. A compartment module member has a plurality of compartments pivotally mounted to the base section and pivotal to a slant position when the cover section is in an opened position.

The dispenser receptacle further receives a stack of first aid items and a spring biases the stack to urge the stack toward a front panel of the cover section. A chute forms an opening through the cover section and an item of the first aid stack is withdrawable through the chute in the closed position. A second dispenser receptacle is carried by the front cover section and the second dispenser receptacle is also accessible through the cover section in the closed position. A spring engages a stack of first aid items received in the second receptacle. The second chute provides an opening so that the first aid item in the second receptacle is withdrawable through the opening. The cover section is preferably transparent or semi-transparent.

A cabinet comprises a first shell and a second shell pivotally joined in the first shell and positionable between an opened and a closed position housing first aid supplies. The first shell forms a restricted opening. A dispenser receptacle is mounted to the first shell and is accessible through the restricted opening in the closed position. The dispenser receptacle has a panel spaced from a front panel of the first shell and a spring biased toward the front panel and having an effective force characteristic of 0.25 to 2.0 lbs. A stack of first aid items is received in the dispenser receptacle and removably retained by the spring so that an item is removable from the receptacle through the restricted opening in the closed position.

A second dispenser receptacle is mounted to the first shell and is accessible through the first shell in the closed position. The second receptacle has a second receptacle panel spaced from a front panel of the first shell and a second spring biased toward the front panel. A second stack of items in the second dispenser receptacle is biased by the spring wherein an item of the second stack is withdrawable through the second restricted opening of the first shell when the first shell is in the closed position.

A dispenser receptacle is carried by the cover section when the cover section is pivotally moved from the closed position to the opened position. The dispenser receptacle is accessible through the opening through the cover section in the closed position. The dispenser receptacle receives a stack of first aid items. An integrated leaf spring engages and biases the stack to urge the stack toward the cover section.

The integrated leaf spring comprises a flat mounting portion and an arm terminating in a contoured portion. The arm is disposed at an acute angle to the mounting portion. The dispenser receptacle has a cover. The mounting portion is mounted to the cover.

The cabinet cover section further has a second opening adjacent the first opening. The second opening has an arcuate shape defined in the top and a generally rectangular shape with a shallow concave portion defined in the side. A second receptacle is carried by the cover section and is accessible through the second opening in the closed position.

A second integrated leaf spring engages a stack of first aid items received in the second receptacle. The second integrated leaf spring has a mounting base and an arm extending at an acute angle and spaced from the base. The arm has a contoured distal end portion. The second receptacle has a cover and the mounting base is mounted to the cover.

### BRIEF DESCRIPTION OF THE DRAWINGS

FIG. 1 is a perspective view of a first aid cabinet;

FIG. 2 is a perspective view of the first aid cabinet of FIG. 1 in an open position;

FIG. 3 is an enlarged perspective view of the first aid cabinet in an open position from a different perspective to that of FIG. 2;

FIG. 4 is an exploded perspective view of the first aid cabinet of FIG. 3;

FIG. 5 is a front elevational view of the first aid cabinet of FIG. 1;

FIG. 6 is a left side elevational view of the first aid cabinet of FIG. 5;

FIG. 7 is a right side elevational view of the first aid cabinet of FIG. 5;

FIG. 8 is a top plan view of the cabinet of FIG. 5;

FIG. 9 is a bottom plan view of the first aid cabinet of FIG. 5;

FIG. 10 is a rear elevational view of the first aid cabinet of FIG. 5;

FIG. 11 is an annotated schematic view illustrating a pair of exteriorly accessible dispenser receptacles of the first aid kit of FIG. 1;

FIG. 12 is an enlarged view of a pivotal multi-compartment member for the first aid cabinet of FIG. 1;

FIG. 13 is a side view, partly in section, of the compartment member of FIG. 12;

FIG. 14A is a front view of a locking plate for the first aid cabinet of FIG. 1;

FIG. 14B is a side perspective view of the locking plate of FIG. 14A;

FIG. 14C is a second perspective view of the locking plate of FIG. 14A;

FIG. 14D is a sectional view through the locking plate of FIG. 14A;

FIG. 15 is a perspective annotated view of the first aid cabinet FIG. 1 schematically illustrating the withdrawal of first aid items from exteriorly accessible dispenser receptacles thereof;

FIG. 16A is a perspective view of a panel for a receptacle for the first aid cabinet of FIG. 1;

FIG. 16B is a diagrammatic plan view of the panel of FIG. 16A;

FIG. 16C is a side diagrammatic view of the panel of FIG. 16A;

FIGS. 17A-17C are respectively a perspective view, a plan view and a side view of a spring employed with the panel of FIG. 16A;

FIG. 18A is a perspective view of a second cover for a second exteriorly accessible receptacle for the cabinet of FIG. 1A;

FIG. 18B is a diagrammatic plan view of the panel of FIG. 18A;

FIG. 18C is a side view of the panel of FIG. 18A;

FIGS. 19A-19C are respectively a perspective view, a side view and a plan view of a spring employed for the dispenser receptacle panel of FIG. 18A;

FIG. 20 is a perspective view of another embodiment of a first aid cabinet;

FIG. 21 is a front elevational view, partly diagrammatic and partly in phantom, of the cabinet of FIG. 20;

FIG. 22 is a side elevational view, partly diagrammatic and partly in phantom, of the cabinet of FIG. 20;

FIG. 23 is a perspective view of yet another embodiment of a first aid cabinet illustrated in an opened position and with dispenser receptacles being illustrated in an opened position;

FIGS. 24A and 24B are respectively a front elevational view and an interior elevational view in a closed position of the first aid cabinet of FIG. 23;

FIGS. 25A-25D are respectively an enlarged respective bottom plan view, perspective bottom view, perspective top view and side view of a dispenser receptacle for the cabinet of FIG. 23;

FIGS. 26A-26C are respectively an enlarged perspective view, top plan view and side schematic view of an integrated base and leaf spring for a dispenser receptacle of FIG. 23;

FIGS. 27A-27D are respectively an enlarged bottom plan view, perspective bottom view, perspective top view and a side view of a cover for another dispenser receptacle of FIG. 23; and

FIGS. 28A-28C are respectively an enlarged perspective view, side schematic elevational view and an integrated base top plan view of leaf spring for another dispenser receptacle of FIG. 23.

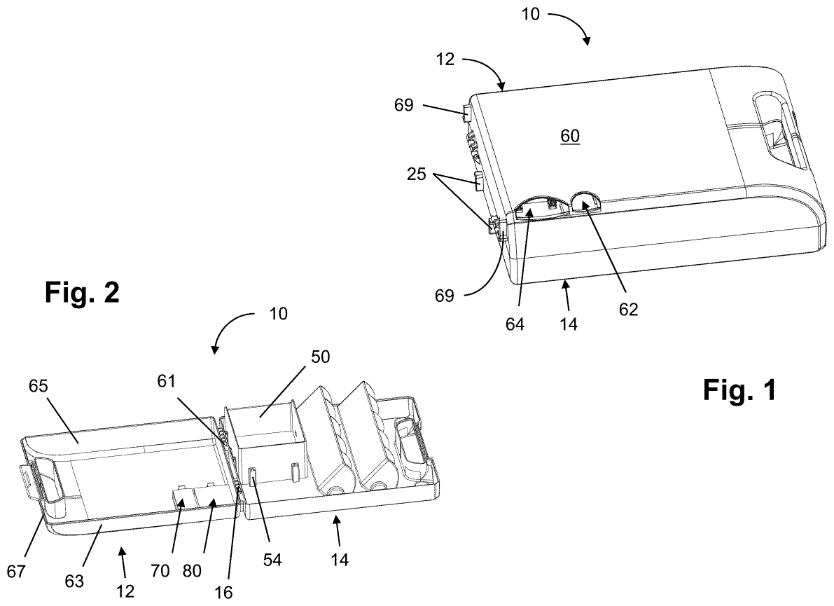

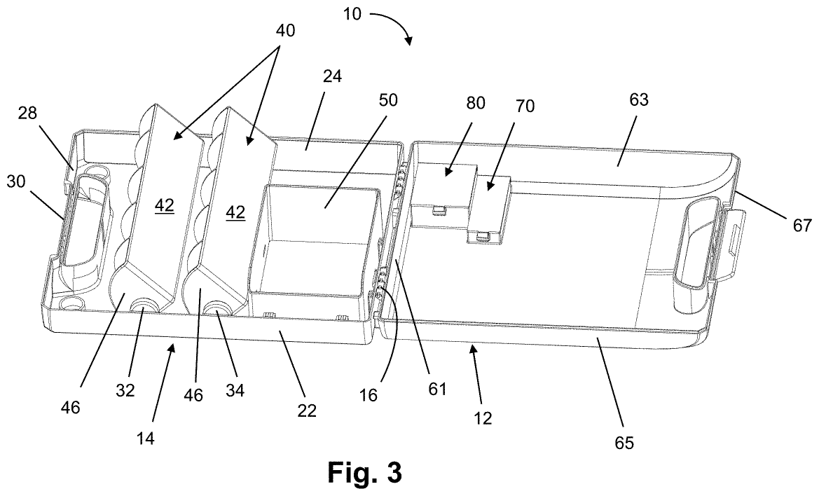

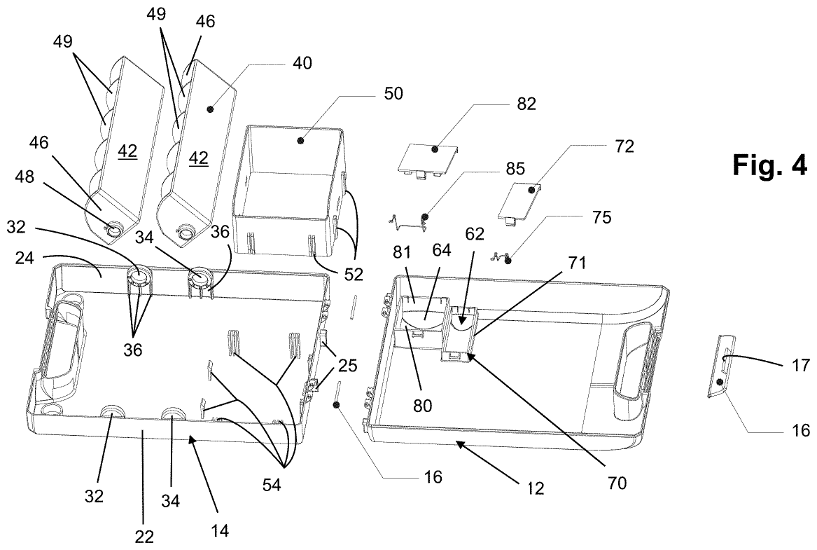

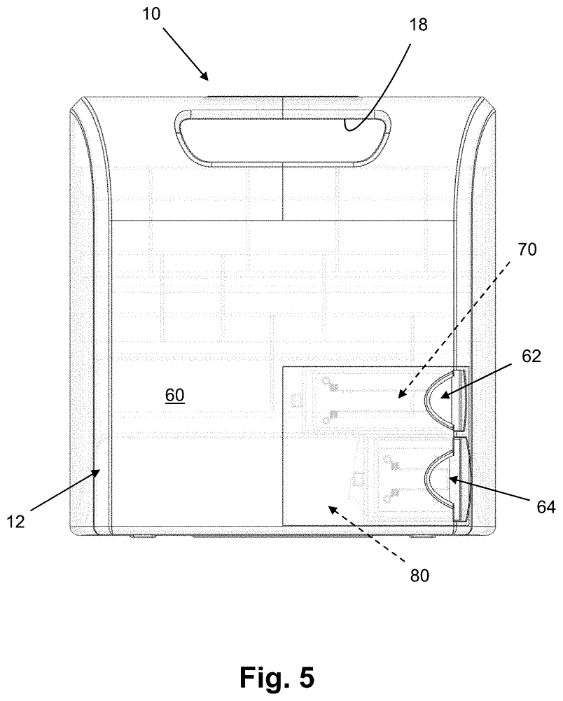

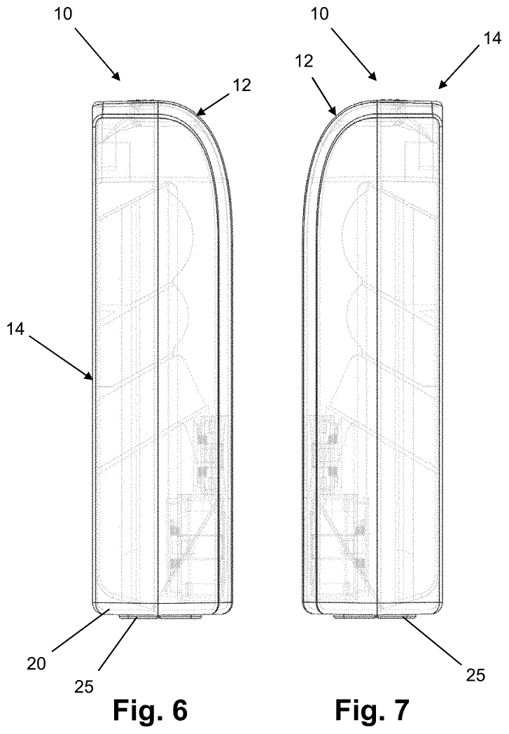

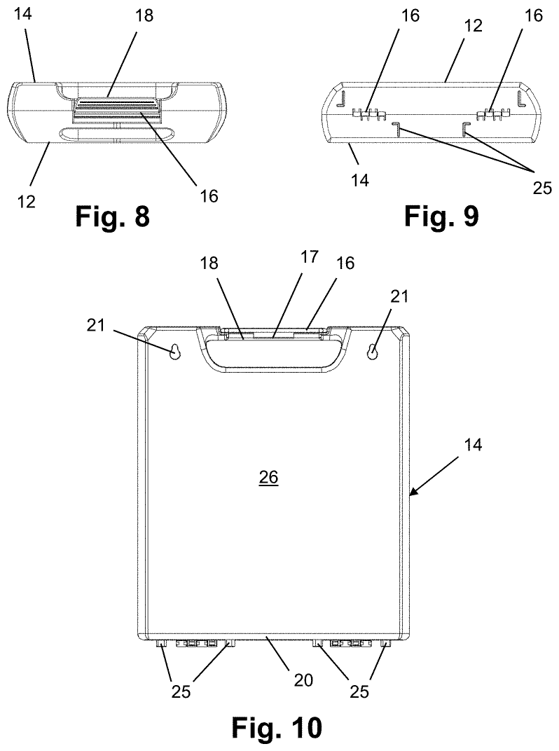

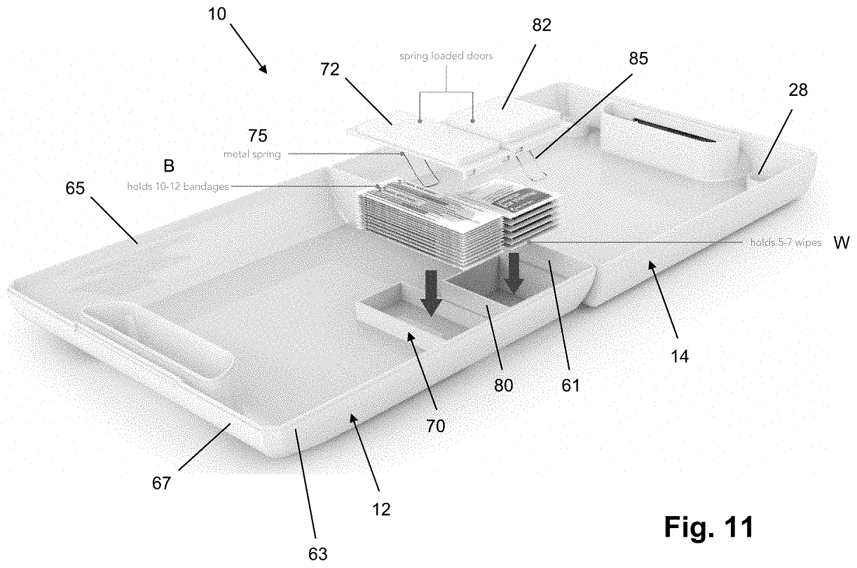

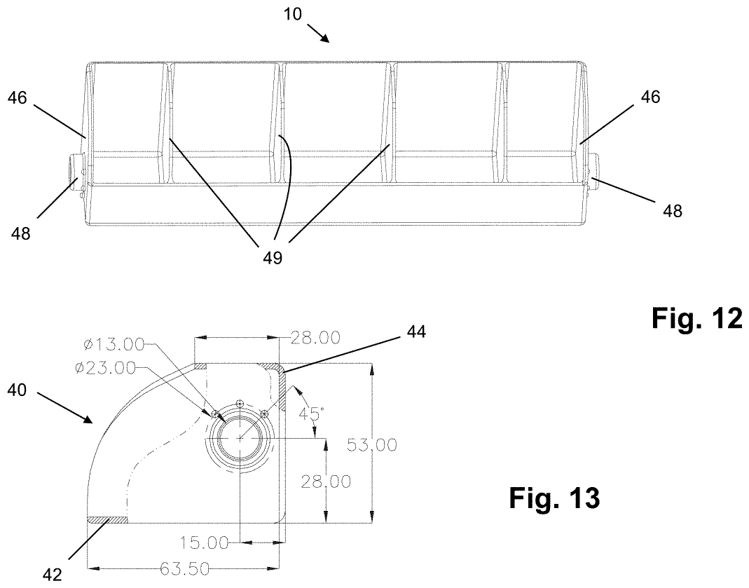

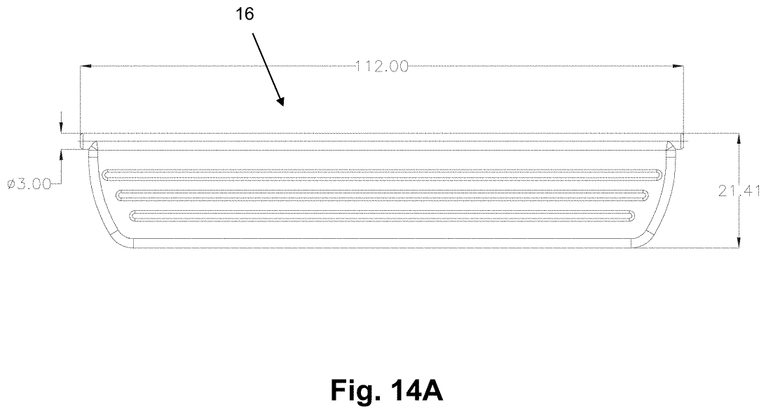

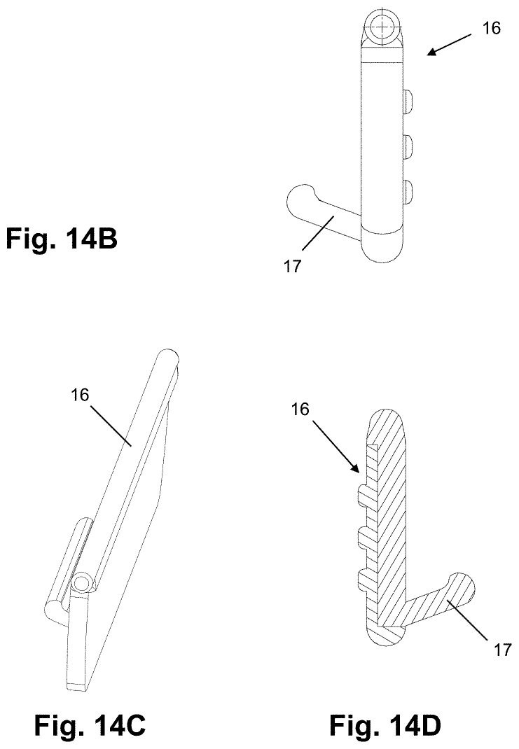

### DETAILED DESCRIPTION

With reference to the drawings wherein like numerals represent like parts throughout the several figures, a first aid cabinet is generally designated by the numeral 10. The first aid cabinet 10 has a pair of front and back shells 12 and 14, respectively, which are pivotally joined by a pair of hinge pins 16. The shells cooperate to form a protective housing for first aid items. The cabinet is formed of a durable material and includes multiple molded components. The cabinet 10 has an aesthetically pleasing contoured external appearance. The cabinet 10 may be translucent, semi-transparent and, in particular, front shell 12 may be transparent in some embodiments. Alternatively, the shells may be pivotally joined about a vertical axis.

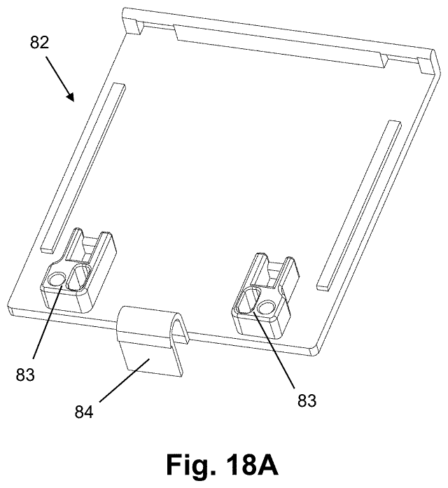

The cabinet 10 is preferably mounted to a wall in an upright orientation. The cabinet 10 also forms an upper recess which is bridged by a contoured handle 18 so that it may be efficiently carried. The cabinet pivots from a closed position to an opened position which readily displays various first aid items. The shells 12 and 14 are secured in a closed position by a pivotal locking plate 16 having a catch 17. See FIGS. 14A-14D. A feature of the cabinet is that in the closed position, the cabinet is configured to provide ready access to various frequently used items stored within the cabinet without opening the cabinet.

Back or rear shell 14 has a semi-rectangular lower panel 20 from which project opposing parallel side panels 22 and 24 and a lateral back panel 26. The opposed panel 28 forms a pair of opposing ends which extend to a medial partially contoured recess for the handle 18. A bridge 30 transverses the recess and partially forms the handle. The panels 20, 22, 24 and 28 preferably have a uniform transverse depth.

The panels 22 and 24 each interiorly mount a pair of opposed inwardly projecting sockets 32 and 34 at intermediate positions along the panels. Ribs 36 extend from the back panel 28 to the sockets to provide reinforcement.

Substantially identical elongated compartment row modules 40 are each pivotally mounted to the sockets. The compartment row modules 40 include a pair of substantially perpendicular shelf elements 42 and 44 and contoured end panels 46. The end panels each mount a projecting cylindrical protuberance 48 which is received in a socket 32 or 34 for limited pivotal rotation therewith. The compartment row modules 40 each have a plurality of intermediate partitions 49 which define a multiplicity of linearly arranged compartments. It will be appreciated that the compartments are each adapted to receive first aid items of various quantities and types. In some embodiments, the compartment row members and the compartments are not identical in shape, size or numbers. Compartments alternatively may be provided as fixed non-pivoting mail-slot structures.

The back panel 26 preferably includes a pair of keyhole slots 21 which facilitate hanging the cabinet on a wall. The lower panel 20 may also include a pair of protruding supports 25 which facilitate support of the cabinet in an upright orientation while the cabinet rests on a horizontal surface.

A generally rectangular box-like receptacle 50 has two pairs of parallel engagement ribs 52 at each of its four sides. The interior of the shell mounts outwardly projecting studs 54. The studs 54 are generally complementary to the ribs 52 and are somewhat resilient to provide a secure engagement when the receptacle is slidably inserted into the shell for engagement against the panel to provide a fixed open receptacle for various first aid items.

The front shell 12 has a front panel 60 which includes and partially forms a pair of dispenser chutes 62 and 64 adjacent a lower side portion thereof. The dispenser chutes 62 and 64 provide restricted interior access to a limited number of items in the cabinet upon closure. The front shell 12 includes a lower panel 61 and side panels 63 and 65 and upper panel 67 which mate along peripheral edges with corresponding panels 20, 22, 24 and 28 in the closed position. Protruding supports 69 extend from the bottom panel 67 and cooperate with supports 25 to support the cabinet on a horizontal surface, if desired.

The dispenser chutes 62 and 64 are preferably located at the front and intersecting side portion of the front shell, but other locations are possible. The dispenser chutes each preferably define an arcuate recessed opening of a small transverse depth to allow for the fingertips of a user to access the outer extremities of the chute. The front shell 12 mounts a pair of rectangular box-like receptacles 70 and 80 which open into a respective chute 62 and 64 to allow selected items in the cabinet to be withdrawn. The receptacles 70 and 80 are fixed to and carried by the front shell 12 and are accessible via the respective chutes 62 and 64.

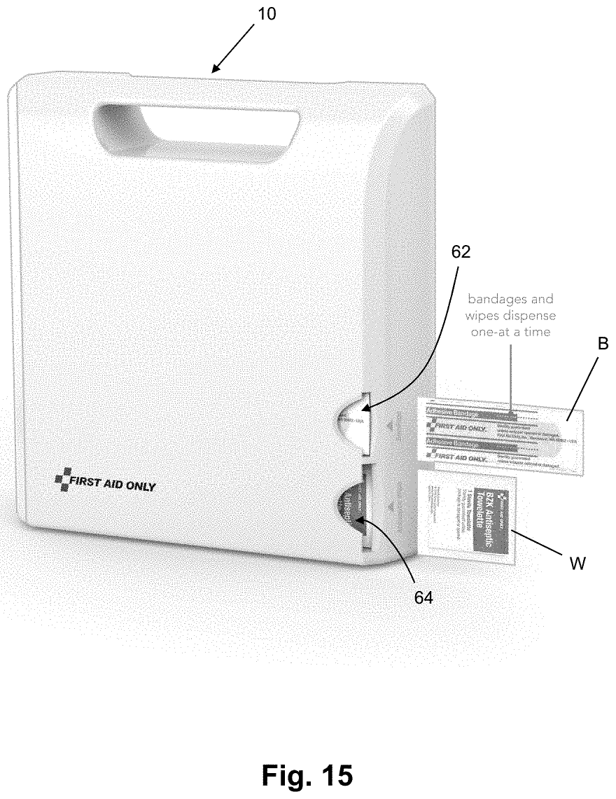

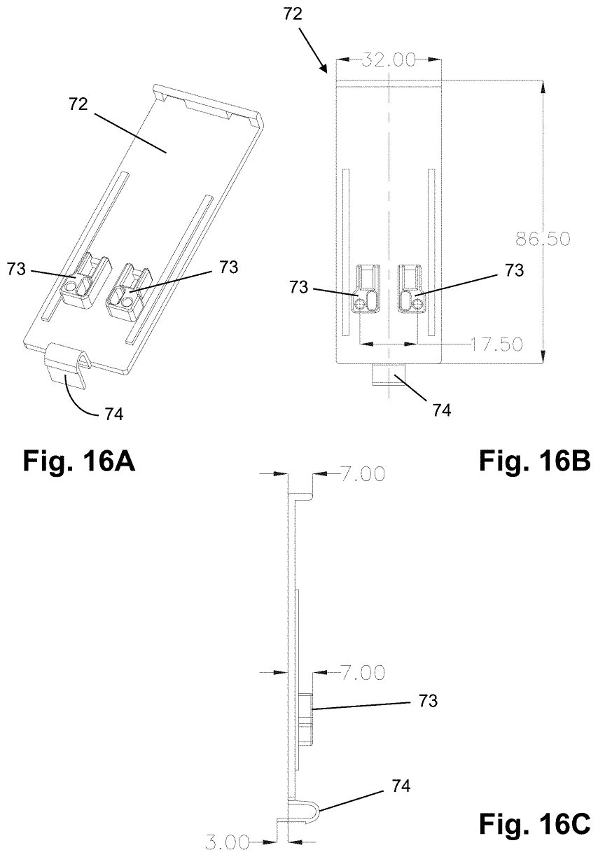

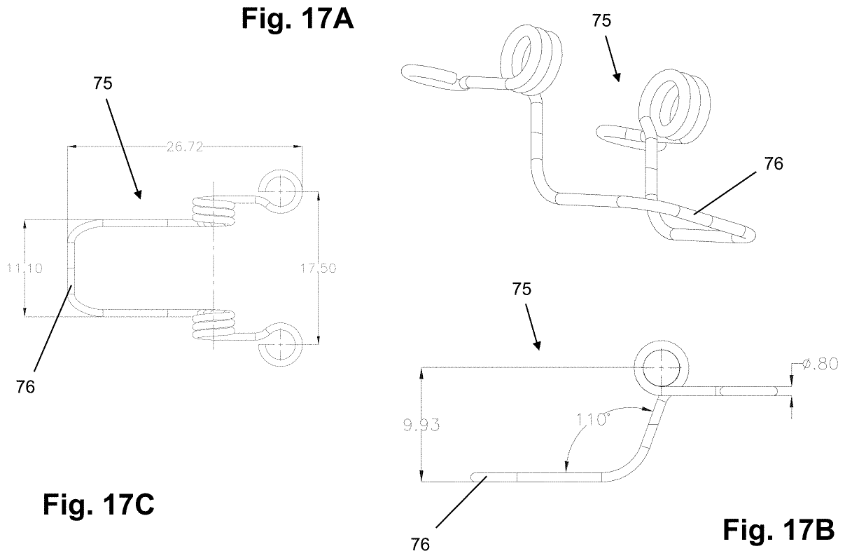

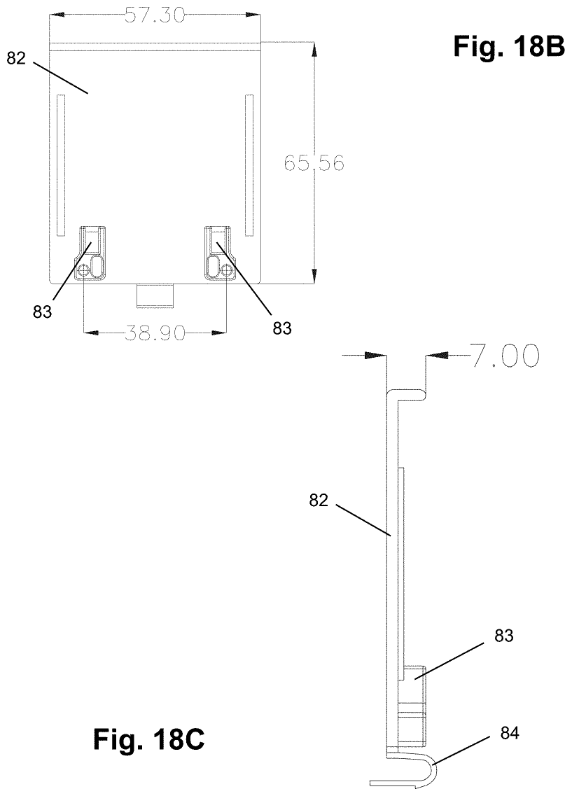

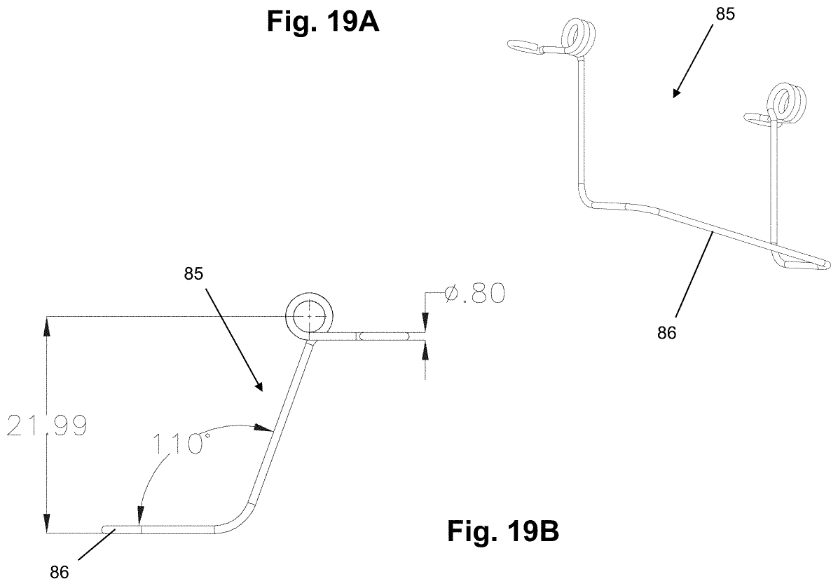

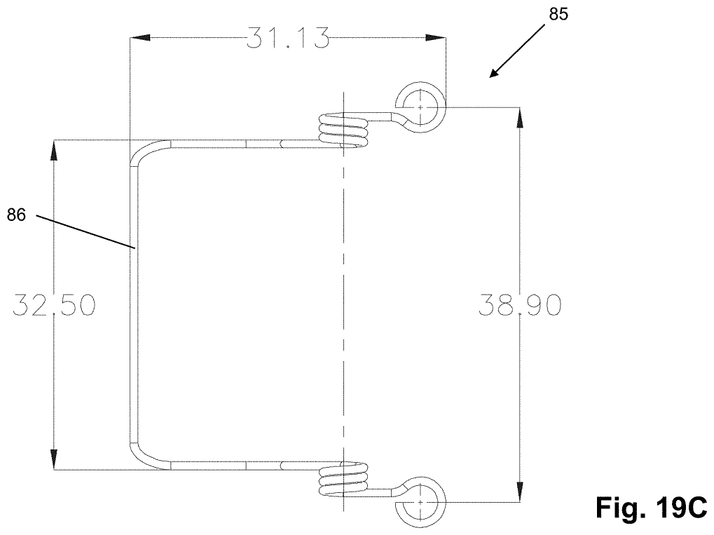

A substantially rectangular enclosure includes a rectangular frame 71 which preferably integrally extends interiorly from the front panel 60. With reference to FIGS. 16A-16D, a back panel 72 has a resilient tab 74 and lip/rim structures which allow the back panel to be snapped into place onto the frame 71 at the interior of the shell. The panel has a pair of slotted bosses 73 which mount a spring 75 (FIGS. 17A-17C). The spring 75 has a quasi-U-shaped medial portion 76 adapted to engage the top of a stack of first aid supplies, such as, for example, 10-12 bandages B (See FIG. 11). The spring is configured and has a force characteristic which is in the range of 0.25 to 2.0 lbs and exerts a pressure of 0.040 to 0.420 psi which allows the stack of supplies to be gently forced against the interior surface of the front panel 60, but allows for relatively easy sequential withdrawal of an item from the stack. As schematically illustrated in FIG. 15, the chute 62 provides an access to the receptacle 70 from the front exterior so that as one item is pulled from the chute, the remaining items in the stack of supplies are urged outwardly toward the front panel 60 so that the succeeding item engages the inside surface of the panel 60.

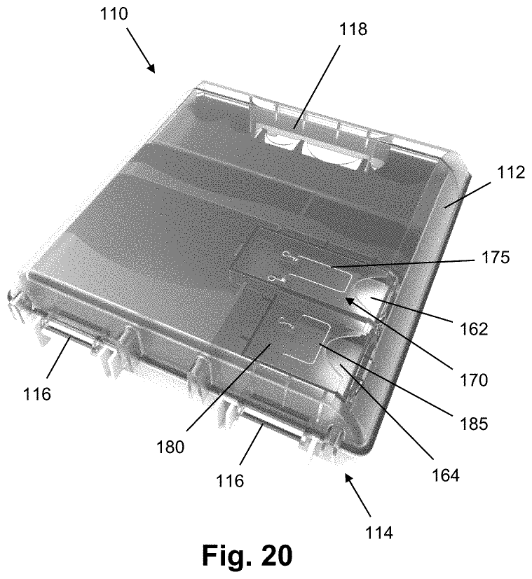

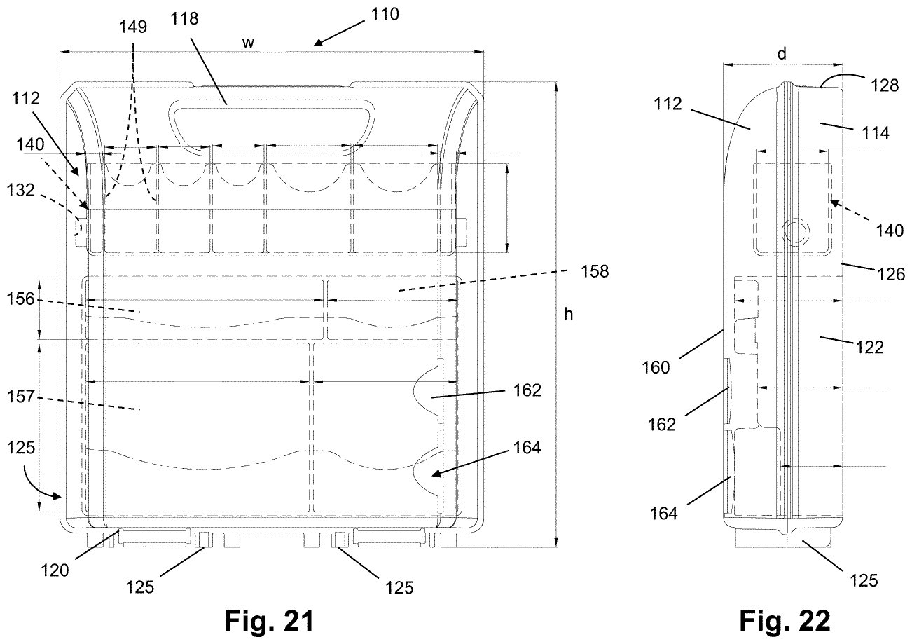

Likewise, a second substantially rectangular receptacle 80 has a substantially rectangular frame 81 integrally extending inwardly from the cover 60. With additional reference to FIGS. 18A-18C, the frame 81 receives a back panel 82 with a resilient tab 84 and lip/rim structures which allow the back to panel 82 be snapped into place onto the frame at the interior of the shell to enclose the receptacle. The panel 82 also includes a pair of slotted bosses 83 which mount a spring 85 (FIGS. 19A-19C). The spring 85 is configured with a quasi-U-shaped medial portion 86 and has a spring force (preferably 0.25 to 2.0 lbs) to provide a sufficient pressure (preferably 0.040 to 0.420 psi) against supplies received in the receptacle. With reference to FIGS. 11 and 15, when a supply item such as wipe W is removed through the chute 64, the next item moves against the inside of the front panel and is urged by the spring to engage the bottom of the stack to retain the stack in the receptacle at the inside surface of the panel 60. The receptacle dimensions may also be varied for a given application.

Only one or more than two dispenser receptacles and chutes may be provided.

The cabinet is preferably provided with first aid supplies which are placed in each of the compartments as well as in the receptacle 50. In addition, wipes W or bandages B are respectively placed in stack-like form in the receptacles, as best illustrated for cabinet 10 in FIG. 11. The cabinet is then pivoted to the closed position. It will be appreciated that wipes and bandages may be withdrawn or dispensed through the dispenser chutes 62 and 64 in the front surface without requiring the cabinet be opened. Naturally, items other than wipes and bandages may be loaded into the dispenser receptacle. The stacks of items are reloadable either through the chutes or by removing the rear panels 72 and 82, loading the stacks and snapping each panel against the corresponding frame to close the receptacle. If additional other first aid items are required, the cabinet may then be pivotally opened, at which time, the compartment row modules 40 will slightly pivot to provide access to various supplies and to provide a ready inspection of the supplies in the cabinet for inventory purposes.

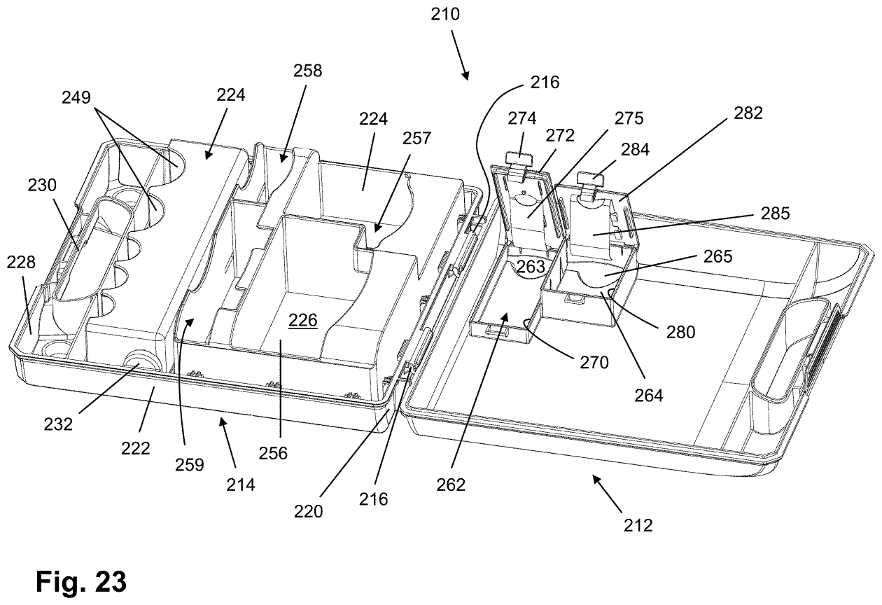

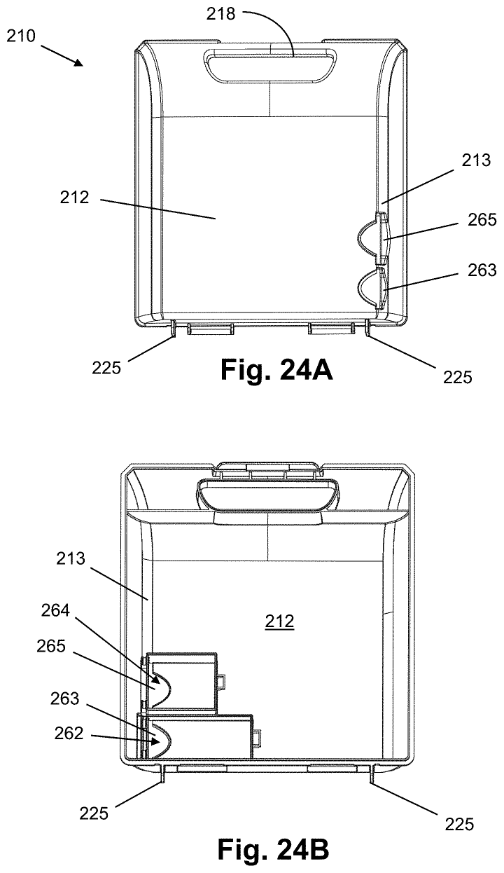

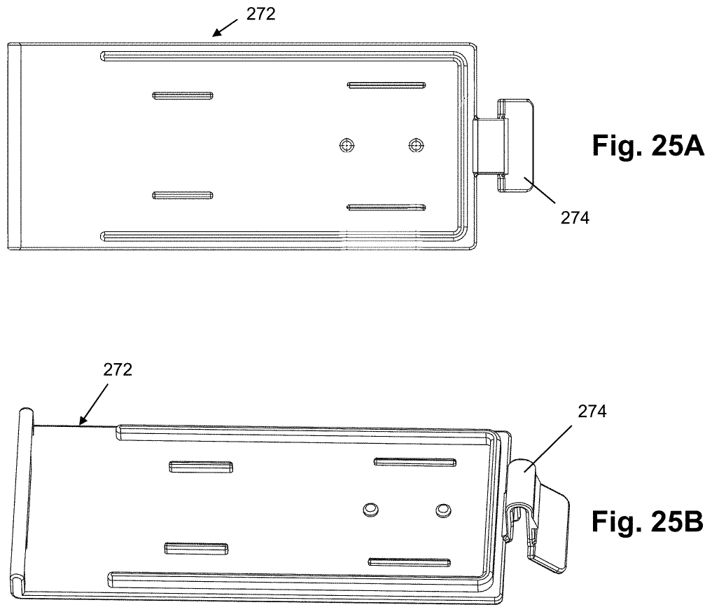

With reference to FIGS. 20-22, another embodiment of a first aid cabinet is generally designated by the numeral 110. The first aid cabinet 110 has a pair of shell-like members 112 and 114 which are pivotally joined by a pair of hinge pins 116. The front shell member 112 is formed of a transparent or semi-transparent material which allows the contents of the cabinet to be viewed when the cabinet is closed. The cabinet 110 is otherwise formed of molded durable material and includes multiple molded components, as well as metal springs 175 and 185, further described below.

The cabinet 110 is preferably mountable to a wall in an upright orientation. The cabinet 110 also forms an upper recess which is bridged by a contoured handle 118 so that it may be efficiently transported. A feature of the cabinet 110 is that in the closed position, the cabinet is again configured to provide ready access to selected items stored within the cabinet without opening the cabinet.

The components of cabinet 110, which corresponds to analogous components of cabinet 10 are designated with corresponding numerals preceded by the numeral 1. The height h, width w and depth d for one embodiment is approximately 298 cm, 271 cm and 76 cm, respectively.

The rear shell 114 has a semi-rectangular lower panel 120 from which project opposing parallel side panels 122 and 124 and the lateral back panel 126. The upper opposed panel 128 forms a pair of opposing ends which extend to a medial partially contoured recess for the handle 118. A bridge 130 traverses the recess and partly forms the handle. The panels 120, 122, 124 and 128 have a generally uniform transverse depth as illustrated in FIG. 20.

The cabinet 110 has a single pivotal compartment module 140 which extends from a pair of opposed inwardly projecting sockets 132 and 124. Compartment row module 140 has a plurality of intermediate partitions 149 which define a multiplicity of compartments. Each of the compartments is adapted to receive first aid items of various quantities and types. The compartment members and the compartments may not be identical in shape, size or number. In the illustrated embodiment, some compartments have identical shapes. For some embodiments, all compartments are fixed and have no pivotal compartment modules.

The back panel 126 preferably includes a pair of keyhole slots (not illustrated) which facilitates hanging the cabinet on a wall. The bottom panel 120 may also include a pair of protruding supports 125 to facilitate support of the cabinet in an upright orientation while the cabinet rests on a horizontal surface.

Generally rectangular open receptacles 156, 157 and 158 are provided for various first aid items. Each receptacle 156, 157 and 158 also preferably includes a frontal contoured skirt-like retainer which allows supplies to be retained in a receptacle. Other receptacles of various sizes and shapes may be formed in the rear shell and secured therewith.

The front cover 112 includes a front panel 160 which mounts a pair of dispenser chutes 162 and 164. The dispenser chutes provide limited interior access to a limited number of items in the cabinet upon closure. The front shell 114 mounts a pair of rectangular receptacles 170 and 180 having interior rear panels which open into a respective chute 162 and 164 to allow selected items in the cabinet to be withdrawn.

Receptacles 170 and 180 have the same structural elements and functions as receptacles 70 and 80, respectively. Enclosure 170 includes a back panel or cover with tabs 174 which allow the panel to be snapped into place onto a rectangular frame which integrally extending from the shell. The panel has a pair of bosses which mount a spring 175. The spring has a quasi-U-shaped medial portion adapted to engage a stack at the top of a first aid supply such as wipes. The spring 175 is configured to and has a force characteristic in the range of 0.25 to 2.0 lbs and allows a stack of supplies to be gently forced against the front panel but allows for withdrawal of an item from the top of the stack. As best illustrated in FIG. 15, for chute 62, chute 162 provides access to the receptacle from the front exterior so that a supply is slidably pulled from the chute. A stack of supplies is urged downwardly against the rear panel.

Likewise, a second receptacle 180 receives a rear panel or cover with tabs 184 which allow the cover to be snapped into place at the interior of the shell to enclose the receptacle. The receptacle also includes a pair of slotted bosses which mounts a spring 185. The spring is configured for the quasi-U-shaped medial portion and has a spring force of approximately 0.25 to 2.0 lbs to provide sufficient pressure (approximately 0.040 to 0.420 psi) against the supplies received in the receptacle when a supply item is removed through the chute.

Cabinet 110 is preferably provided with first aid supplies placed in each of the compartments as well as in the receptacle. Bandages and wipes are respectively placed in stack-like form in the receptacles. The cabinet may then be pivoted to the closed position. It will be appreciated that the wipes and bandages may be withdrawn through the chutes and the front surface without requiring the cabinet to be opened. When the cabinet is opened, the bandages and wipes are secured to the front cover by the enclosures 170 and 180 which move with the front shell or cabinet cover. Naturally, other items may be placed in the receptacles.

When the cabinet 110 is pivotally opened, a compartment module will slightly pivot to provide access to various supplies, and the other compartments will be readily accessible so that the supplies may be removed. The transparent or semi-transparent nature of the cover also provides for a ready inspection of the supplies in the cabinet for inventory purposes.

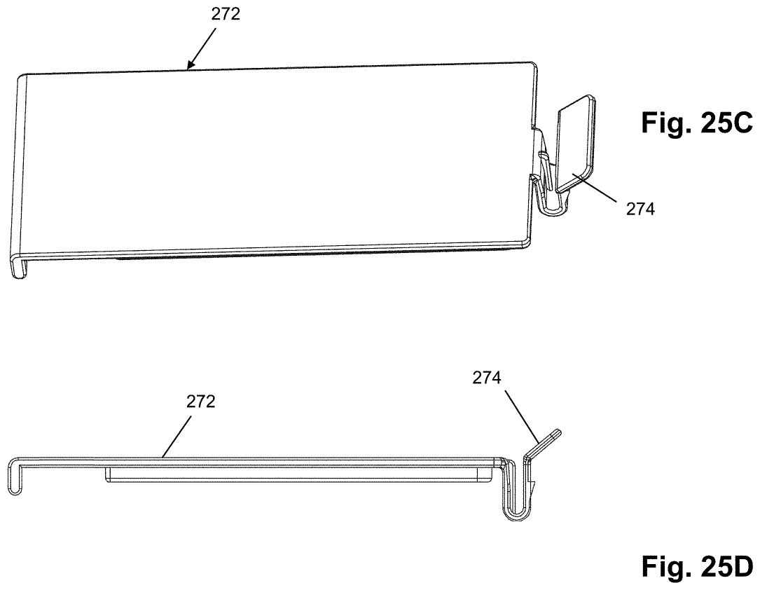

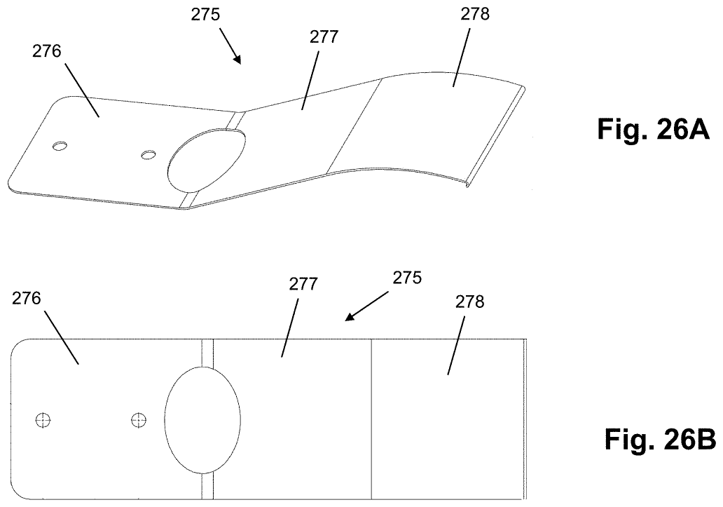

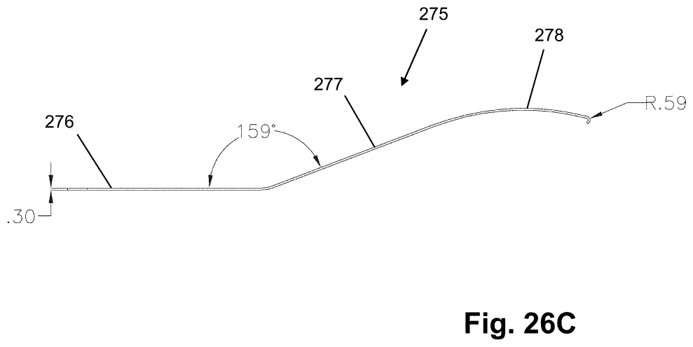

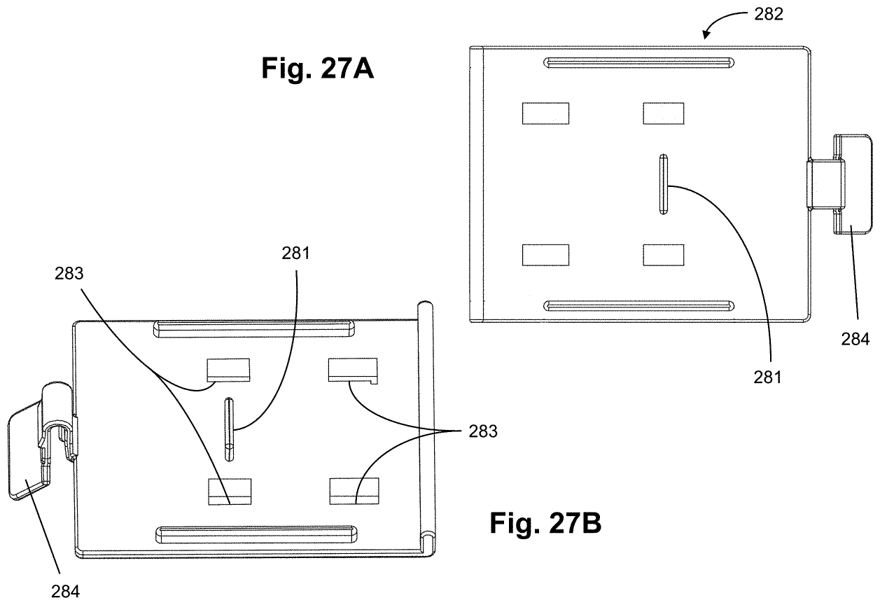

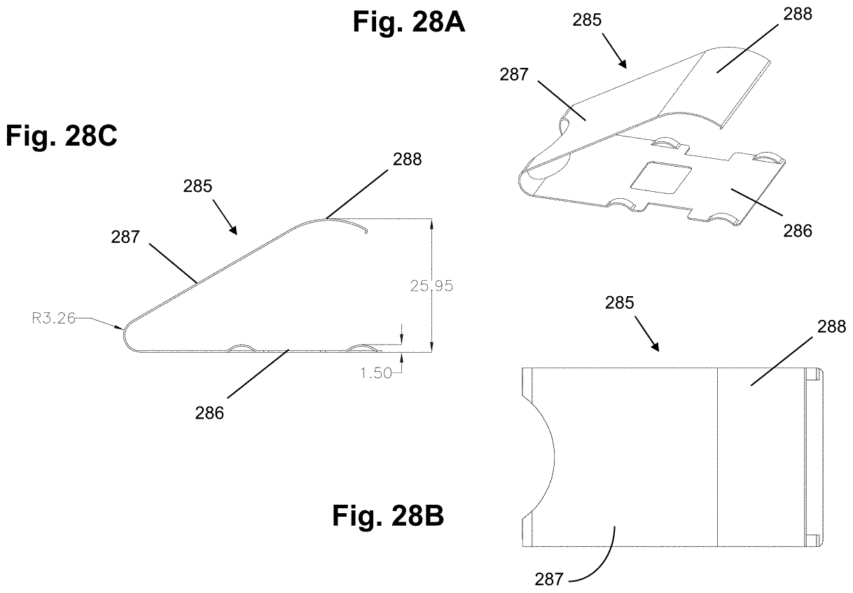

With reference to FIGS. 23-28C, another embodiment of a first aid cabinet is generally designated by the numeral 210. The first aid cabinet 210 has a pair of shell-like members 212 and 214 which are pivotally joined by a pair of hinge pins 216. The front shell member 212 is formed of a transparent or semi-transparent material which allows the contents of the cabinet to be viewed when the cabinet is closed. The cabinet 210 is otherwise formed of molded durable material and includes multiple molded components, as well as metal integrated base and leaf springs 275 and 285 illustrated in FIGS. 26A-26C and FIGS. 28A-28C, respectively, and further described below.

The cabinet 210 is preferably mountable to a wall in an upright orientation. The cabinet 210 also forms an upper recess which is bridged by a contoured handle 218 so that it may be efficiently transported. A feature of the cabinet 210 is that in the closed position, the cabinet is again configured to provide ready access to selected items stored within the cabinet without opening the cabinet.

The components of cabinet 210, which corresponds to analogous components of cabinet 10 are designated with corresponding numerals preceded by the numeral 2. The height h, width w and depth d for cabinet 210 in one preferred embodiment is approximately 298 cm, 271 cm and 76 cm, respectively.

The rear shell 214 has a semi-rectangular lower panel 220 from which project opposing parallel side panels 222 and 224 and the lateral back panel 226. The upper opposed panel 228 forms a pair of opposing ends which extend to a medial partially contoured recess for the handle 218. A bridge 230 traverses the recess and partly forms the handle. The panels 220, 222, 224 and 228 have a generally uniform transverse depth as illustrated in FIG. 23.

The cabinet 210 has a single pivotal compartment module 240 which extends from a pair of opposed inwardly projecting sockets. Compartment row module 240 has a plurality of intermediate partitions 249 which define a multiplicity of compartments. Each of the compartments is adapted to receive first aid items of various quantities and types. The compartment members and the compartments may not be identical in shape, size or number. In the illustrated embodiment, some compartments have identical shapes. For some embodiments, all compartments are fixed and have no pivotal compartment modules.

The back panel 226 preferably includes a pair of keyhole slots (not illustrated) which facilitates hanging the cabinet on a wall. The bottom panel 220 may also include a pair of protruding supports 225 to facilitate support of the cabinet in an upright orientation while the cabinet rests on a horizontal surface.

Generally rectangular open receptacles 256, 257, 258 and 259 are provided for various first aid items. The receptacles 256, 257, 258 and 259 also preferably includes a frontal contoured skirt-like retainer which allows supplies to be retained in a receptacle. Other receptacles of various sizes and shapes may be formed in the rear shell and secured therewith.

The front cover 212 includes a front panel 260 which mounts a pair of dispenser chutes 262 and 264 having respective openings 263 and 265. The openings each have an arcuate shape in the front and a shallow rectangular slightly concave shape in a top side portion at side 213. The dispenser chutes provide expedited access to a limited number of items in the cabinet upon closure. The front shell 214 mounts a pair of rectangular receptacles 270 and 280 having interior rear panels or covers. The receptacles 270 and 280 open at one end to form the respective chute 262 and 264 to allow selected items in the cabinet to be withdrawn through the respective openings 263 and 265 while protecting the selected items when the cabinet is closed.

Receptacles 270 and 280 have substantially the same structural elements and functions as receptacles 70 and 80, respectively. Enclosure 270 includes a back panel or cover 272 with tabs and a resilient latch tab 274 which allows the panel to be snapped into place onto the rectangular frame integrally extending from the shell. The panel 272 has a pair of bosses which mounts an integrated base and leaf spring 275.

With reference to FIGS. 26A-26C, the integrated leaf spring 275 has a flat anchor or base portion 276, an angled extension 277 extending approximately 159° in a rest state and a shallow contoured distal end portion 278. The angled extension 277 is adapted to engage a stack at the underside of a first aid supply such as bandages. The spring is configured to and has a force characteristic in the range of 0.25 to 2.0 lbs and allows a stack of supplies to be gently forced against the front panel but allows for ready withdrawal through opening 263 of an item from the top of the stack. The base portion 276 is mounted to the back or cover panel 272, and the contoured end portion 278 is disposed adjacent the side of the front panel. As best illustrated in FIG. 15, for chute 62, chute 262 provides access to the receptacle from the front exterior so that a supply item is slidably sequentially pulled from the chute through the opening 263. The stack of supplies is urged toward the front panel.

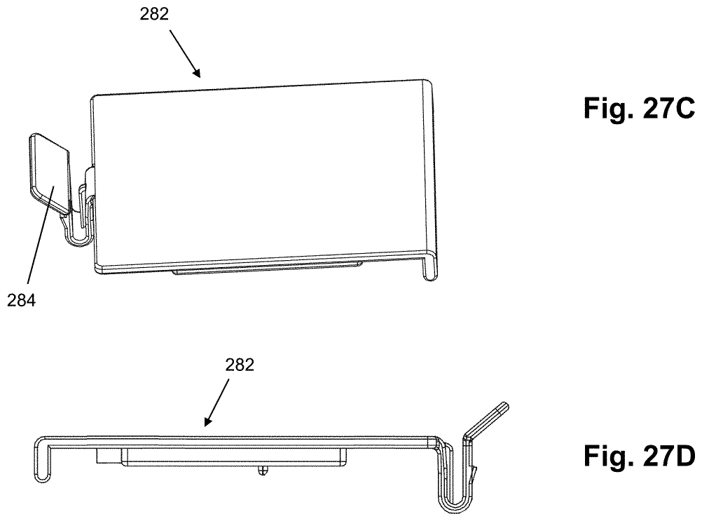

Likewise, with reference to FIGS. 23, 27A-27D, a second receptacle 280 receives a rear panel cover 282 with tabs and a resilient latch 284 which allows the cover to be snapped into place at the interior of the shell to enclose the receptacle. The cover plate 282 also includes a tab 281 and two pairs of slotted bosses 283 which mounts an integrated base and leaf spring 285. With reference to FIGS. 28A-28C, the integrated leaf spring 285 is configured in an angled form having a flat anchor base 286 and a reverse engagement arm 287 at an acute angle and having a shallow contoured distal end 288. The base 286 is mounted to the underside of the rear panel 282, and the contoured distal end 288 is adjacent the side panel of the cover. The integrated leaf spring 285 has a spring force of approximately 0.25 to 2.0 lbs to provide appropriate pressure (approximately 0.040 to 0.420 psi) against the supplies (preferably antiseptic wipes) received in the receptacle when a supply item is removed through the chute opening 265.

Cabinet 210 is preferably provided with first aid supplies placed in each of the compartments as well as in the receptacle. Bandages and wipes are respectively placed in stack-like form in the receptacles 270 and 280. The cabinet may then be pivoted to the closed position. It will be appreciated that the wipes and bandages may be easily and quickly accessed and sequentially withdrawn in expedited fashion through the chutes without requiring the cabinet to be opened. The openings 263 and 265 and the respective leaf springs 275 and 285 are cooperatively configured to provide the latter feature and to protect the first aid items and maintain same in an orderly fashion. When the cabinet is opened, the bandages and wipes are secured to the front cover by the dispenser receptacles 270 and 280 which move with and are carried by the front shell or cabinet cover. Naturally, other items may be placed in the receptacles.

While preferred embodiments of the foregoing have been set forth for purposes of illustration, the foregoing description should not be deemed a limitation of the invention herein. Accordingly, various modifications, adaptations and alternatives may occur to one skilled in the art without departing from the spirit and the scope of the present invention.

1. A cabinet comprising:
a base section;
a cover section pivotally joined to the base section, and positionable between an opened and a closed position forming a housing, said cover section defining an opening having an arcuate shape in a top portion and a generally rectangular shape with a shallow concave portion in a side portion of the cover section; and
a dispenser receptacle mounted to said cover section and carried by said cover section when the cover is pivoted from the closed position to the opened position and accessible through said opening in the cover section in the closed position and having an integrated leaf spring disposed at an acute angle and biased toward an interior surface of said cover section.

2. The cabinet of claim 1 wherein said dispenser receptacle further receives a stack of first aid items and the spring biases against said stack to urge the stack toward a front panel portion of said cover section.

3. The cabinet of claim 2 wherein said dispenser receptacle forms a dispenser chute accessible through the opening cover section and an item of said stack is sequentially withdrawable through said opening.

4. The cabinet of claim 1 further comprising a second dispenser receptacle mounted to said cover section, said second dispenser receptacle being accessible through the cover section in the closed position.

5. The cabinet of claim 4 further comprising a second integrated leaf spring which biases a stack of first aid items received in said second dispenser receptacle to urge the stack toward a front panel portion of said cover section.

6. The cabinet of claim 5 wherein said second receptacle forms a second dispenser chute accessible through a second opening in said cover section so that a first aid item in said second dispenser receptacle is withdrawable through said second opening.

7. The cabinet of claim 1 wherein said base section and said cover section cooperatively form a recess and a handle traverses along said recess.

8. The cabinet of claim 1 wherein said base section has a panel which defines a pair of keyhole openings.

9. The cabinet of claim 1 further comprising a pivotal locking plate for securing said cabinet in a closed position.

10. The cabinet of claim 1 wherein said cover section is transparent or semi-transparent.

11. A first aid cabinet comprising:
a base section;
a cover section pivotally joined to the base section, and positionable between an opened and a closed position forming a housing for first aid supplies, said cover section having a top and a side and defining an opening having an arcuate shape defined in said top and a generally rectangular shape with a shallow concave portion in said side;
a dispenser receptacle carried by said cover section when said cover section is pivotally moved from the closed position to the opened position and accessible through said opening through said cover section in the closed position; and
a plurality of compartments disposed in said base section and receiving first aid supplies,
wherein said dispenser receptacle further receives a stack of first aid items and an integrated leaf spring engages and biases said stack to urge the stack toward said cover section.

12. The cabinet of claim 11 wherein the integrated leaf spring comprises a flat mounting portion and an arm terminating in a contoured portion and disposed at an acute angle to said mounting portion.

13. The cabinet of claim 12 wherein said dispenser receptacle has a cover and said mounting portion is mounted to said cover.

14. The cabinet of claim 11 wherein said cover section further defines a second opening adjacent said first opening and having an arcuate shape defined in said top and a generally rectangular shape with a shallow concave portion defined in said side and a second receptacle carried by said cover section, said second receptacle being accessible through the second opening in the cover section in the closed position.

15. The cabinet of claim 14 further comprising a second integrated leaf spring which engages a stack of first aid items received in said second receptacle.

16. The cabinet of claim 15 wherein said second integrated leaf spring comprises a mounting base and an arm extending at an acute angle spaced from said base and having a contoured distal end portion.

17. The cabinet of claim 11 wherein said second receptacle has a cover and said base is mounted to said cover.

18. A cabinet comprising:
a first shell;
a second shell pivotally joined to the first shell and positionable between an opened and a closed position to form a housing, said second shell defining a restricted opening with an arcuate portion; and
a dispenser receptacle mounted to said second shell and carried by said second shell wherein said second shell is pivoted from a closed position to an opened position and accessible through said restricted opening in the closed position, said receptacle having a panel spaced from a front panel of said second shell and a leaf spring mounted to said panel and biased toward said front panel and having a force of 0.25 to 2.0 lbs; and
a stack of items received in said dispenser receptacle and engaged by said spring wherein an item is removable from said receptacle through said restricted opening in the closed position.

19. The cabinet of claim 18 further comprising a second receptacle mounted to said second shell and accessible in the closed position through a second opening having an arcuate portion defined in said second shell, said second dispenser receptacle having a second receptacle panel spaced from a front panel of said second shell and a second leaf spring mounted to said second receptacle panel and biased toward said front panel with a second stack of items biased by said second spring, wherein an item of said second stack is withdrawable through a second opening of said second shell when the second shell is in the closed position.

20. The cabinet of claim 19 wherein said second shell has a top portion, said first and second openings each has an arcuate shape in the top portion and a concave shape in the side portion.
